(19) United States
(12) Patent Application Publication
(10) Pub. No.: US 2020/0306302 A1
(43) Pub. Date: Oct. 1, 2020

(54) TREATING AND INHIBITING LEUKEMIA WITH NK-92 CELLS

(71) Applicant: NantKwest, Inc., San Diego, CA (US)

(72) Inventors: Hans G. Klingemann, Boston, MA (US); Tien Lee, San Diego, CA (US)

(73) Assignee: NantKwest, Inc., San Diego, CA (US)

(21) Appl. No.: 16/636,266

(22) PCT Filed: Aug. 3, 2018
(86) PCT No.: PCT/US2018/045146

§ 371 Date: Feb. 3, 2020

Related U.S. Application Data
(60) Provisional application No. 62/541,511, filed on Aug. 4, 2017.

(51) Int. Cl. A61K 35/17; A61P 35/02; C12N 5/0783

(52) U.S. Cl. CPC A61K 35/17; A61K 45/06; C12N 5/0646; A61P 35/02

(57) ABSTRACT

Described herein are methods for treating or preventing leukemias with NK-92 cells. In particular, provided are methods of treating or preventing leukemias by administering to a patient one or more doses of NK-92 cells for killing remnant (also referred to as residual) leukemia cells and/or leukemia stem cells. In various embodiments, NK-92 cells are administered to a patient to treat and/or prevent leukemia that is refractory or resistant, or has relapsed in a patient who is recovering from treatment for leukemia under conventional therapies.

### FIELD OF THE INVENTION

The present disclosure relates to methods for treating, preventing or inhibiting relapse of leukemia with NK-92 cells. The present disclosure further relates to methods for targeting and eliminating leukemia stem cells using NK-92 cells.

### BACKGROUND OF THE INVENTION

Hematologic malignancies, such as leukemia, are among the most common cancers worldwide. Leukemia, such as acute myeloid leukemia (AML), is one of the most common pediatric malignancies and remains the leading cause of death from a disease in children. In adults, hemic malignancies account for about 10% of all cancers. While chemotherapy together with targeted therapy remains the mainstay of treatment, leukemia and other blood-borne cancers have a high rate of relapse. For example, it has been reported that approximately 40-60% of patients treated for AML with conventional therapy evidenced relapse following remission induction chemotherapy. In other instances, it has been reported that pediatric patients suffering from acute lymphoblastic leukemia (ALL) evidenced a 20% relapse rate following remission induction chemotherapy. Choi et al., Blood 110(1):632-639 (2007). Bone marrow transplantation presently offers the only means of cure for patients with relapse of leukemia.

It is believed that in such leukemias, relapse occurs because conventional therapy (e.g., chemotherapy and/or radiation therapy) fails to kill off all of the aberrant cells and especially aberrant stem cells involved in the disease. However, conventional therapy as the primary therapy can be effective in eliminating substantially all of the leukemia cells in the patient.

Current therapy for leukemia includes, by way of non-limiting example, chemotherapy, hormonal therapy, and/or radiation treatment to eradicate the aberrant cells in a patient (see, for example, Stockdale, 1998, Medicine, vol. 3, Rubenstein and Federman, eds., Chapter 12, Section IV). Recently, such therapy has also involved biological therapy or immunotherapy. It is well known in the art, however, that all of these approaches pose significant drawbacks for the patient.

Immunotherapy involves the use of certain cells of the immune system that have cytotoxic activity against particular target cells, i.e., leukemia cells. For example, endogenous natural killer (NK) cells are cytotoxic lymphocytes that constitute a major component of the innate immune system. NK cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. Endogenous NK cells used for this purpose are isolated from the peripheral blood lymphocyte (“PBL”) fraction of blood from the subject, cultivated in cell culture in order to obtain sufficient numbers of cells, and then the cells are re-infused into the subject. NK cells have been shown to be somewhat effective in both ex vivo therapy and in vivo treatment.

NK-92 cells have previously been evaluated as a therapeutic agent in the treatment of certain cancers. Unlike endogenous NK cells. NK-92 cells are a cancer cell line which was discovered in, and obtained from, the blood of a subject suffering from a non-Hodgkin's lymphoma. NK-92 cells lack the major inhibitory receptors that are displayed by normal NK cells, but retain the majority of the activating receptors. Characterization of the NK-92 cell line (Gong et al., 1994; Yan et al., 1998) revealed that NK-92 cells are cytotoxic to a significantly broader spectrum of leukemia cell types than are NK cells, and further that they often exhibit higher levels of cytotoxicity toward these targets. NK-92. cells do not, however, attack normal cells nor do they elicit an immune rejection response.

Accordingly, there remains an ongoing need for new methods of treating or preventing leukemia in patients, especially where the leukemia is refractory or resistant to conventional therapies or in which the leukemia has relapsed or is at risk of relapse following treatment with conventional therapies.

### SUMMARY OF THE INVENTION

Described herein is the use of conventional therapy, such as chemotherapy and/or radiation therapy, in combination with NK-92 cell immunotherapy for the treatment of leukemia. In one aspect, the present disclosure encompasses methods of treating or preventing relapse of leukemia in a patient in recovery from leukemia by administering to a patient one or more doses of NK-92 cells for killing remnant (also referred to as residual) leukemia cells simultaneous with or following conventional therapy. In various embodiments, NK-92 cells are administered to a patient to prevent relapse of leukemia that is refractory or resistant. In other embodiments, NK-92 cells are administered to a patient who has relapsed or is at risk of relapsing after the patient has received treatment for leukemia under conventional therapies. In still other embodiments, one or more doses of NK-92 cells are administered to the patient, optionally in combination with at least one anti-leukemic agent.

Without being bound by theory, it is believed that at least a subset of cancer relapse after conventional therapy is due to the inability of the therapy to fully eradicate cancer stem cells in a patient who otherwise is considered to be in remission. The cancer stem cells may remain in the patient for a period of time after treatment, and gradually multiply such that the cancer returns (relapse). For example, leukemia stem cells may remain in a patient despite remission and eventually cause relapse of the leukemia. Endogenous NK cells target and kill cancer stem cells. It is believed that treatment of a patient with NK-92 cells will target the cancer stem cells and eradicate (or nearly eradicate) the cancer stem cells in order to prevent or delay relapse of the cancer.

In one aspect, a method for treating remnant leukemia cells in a patient previously treated for leukemia is provided, method comprising administering to said patient NK-92 cells in an amount sufficient to kill a population of remnant leukemia cells remaining in the patient following conventional therapy for said leukemia.

In some embodiments, prior to administering the NK-92 cells to the patient, the population of remnant leukemia cells are present in the patient at a level that is less than about 10% of the level of leukemia cells that was detected in the patient prior to the treatment for leukemia. In some embodiments, the remnant leukemia cells comprise leukemic stem cells. In some embodiments, the remnant leukemia cells comprise marrow cell precursors of lymphocytes, red blood cells, white blood cells or platelets. In some embodiments, the conventional therapy comprises one or more of chemotherapy, radiotherapy, hormone treatment or a bone marrow transplant. In some embodiments, the remnant leukemia cells are resistant to conventional therapy.

In one embodiment, NK-92 cells are administered to the patient concurrently with conventional cancer treatment. In one embodiment, NK-92 cells are administered to the patient following conventional cancer treatment, e.g., immediately following treatment and/or while the patient is in remission. In one embodiment, NK-92 cells are administered to the patient upon the first sign(s) of relapse of the leukemia. In one embodiment, NK-92 cells are administered to the patient upon relapse of the leukemia.

In particular, for those leukemia patients who appear to be cancer free by virtue of conventional therapy, NK-92 cell immunotherapy is provided to eradicate any remaining undetected cancer cells, including aberrant stem cells which are recalcitrant to conventional therapy, and/or to prevent relapse of the disease. In such methods, it is contemplated that the incidence of relapse will be significantly reduced as compared to the use of either NK-92 immunotherapy or conventional therapy as the primary and only therapy.

In one aspect, provided is a method for eradicating remaining leukemia cells, including aberrant stem cells, in a patient who has undergone primary treatment of leukemia with conventional therapy. In one embodiment, the method comprises identifying a patient population having undergone primary treatment of leukemia with conventional therapy, wherein that population is believed to be in recovery; and administering an effective amount of NK-92. cells to the patient so as to eradicate all or substantially all (e.g., at least 50%, 60%, 70%, 80%, 85%, 90%, 91, %, 92%, 93%, 94%, 95%, 96%, 97%, 98%, 99%, 99.5%, or 99.9%) of the remaining leukemia cancer cells, including aberrant stem cells.

In another aspect, there is provided a method for inhibiting relapse of leukemia in a patient who is in recovery from leukemia, where said method comprises administering an effective amount of NK-92 cells to said patient, so as to eradicate all or nearly all remaining leukemia cells, including aberrant stem cells which, if present, could cause relapse of the leukemia in the patient. In some embodiments, the method comprises administering to the patient one or more doses of NK-92 cells in an amount sufficient to inhibit relapse of leukemia in the patient. In one embodiment, relapse of leukemia in the patient is prevented. In one embodiment, relapse of leukemia in the patient is delayed. In some embodiments, the relapse of leukemia in the patient is inhibited for at least about three months following the administration of the NK-92 cells. In some embodiments, the leukemia is a lymphocytic leukemia or a myelogenous leukemia.

In another aspect, there is provided a method for treating relapsed leukemia in a patient who underwent conventional therapy for leukemia as the primary therapy, wherein one or more doses of NK-92 cells are administered to the patient. In some embodiments, the method is for treating relapse of leukemia in a patient previously in recovery from leukemia, the method comprising administering to the patient one or more doses of NK-92 cells in an amount sufficient to treat the relapsed leukemia in the patient. In one embodiment, the patient is administered at least one anti-leukemic agent in combination with the NK-92 cell treatment.

In another aspect, there is provided a method for treating leukemia in a patient who underwent conventional therapy for leukemia, the method comprising administering to the patient one or more doses of NK-92 cells in a therapeutic amount as an alternative to chemotherapy. In some embodiments, the one or more doses of NK-92 cells administered to the patient serves as the primary therapy after a relapse of leukemia in the patient.

In some embodiments, the NK-92 cells are unmodified NK-92 cells. In some embodiments, the NK-92 cells are genetically modified NK-92 cells. In some embodiments, the NK-92 cells are irradiated prior to being administered to the patient. In some embodiments, the NK-92. cells secrete interleukin-2.

In another aspect, a method is provided for treating a patient who is genetically predisposed to leukemia, where the method comprises administering to the patient one or more doses of NK-92 cells, such that the one or more doses are sufficient to treat the patient.

In any of the aspects and embodiments described herein, conventional therapy includes, without limitation, one or more of chemotherapy, radiotherapy, hormone treatment, bone marrow transplant, biological therapy, or immunotherapy (other than NK-92 therapy).

In another aspect, provided is a pharmaceutical composition comprising a therapeutic dose of NK-92 cells for the treatment of leukemia.

In another aspect, provided is a pharmaceutical composition comprising a prophylactic dose of NK-92 cells for treating a patient that is genetically predisposed to leukemia, such as a myelodysplastic syndrome.

In another aspect, provided is a kit comprising a prophylactic dose of NK-92 cells for the effective treatment of a patient that is genetically predisposed to leukemia, such as a myelodysplastic syndrome, or to inhibit relapse of leukemia in a patient in recovery.

In another aspect, use of a composition described herein for treating a disease is provided. In some embodiments, a pharmaceutical composition comprising a therapeutic dose of NK-92 cells is provided for use as a medicament for treating a disease. In some embodiments, a pharmaceutical composition comprising a therapeutic dose of NK-92 cells is provided for use in the treatment of a disease. In some embodiments, a pharmaceutical composition comprising the NK-92 cells described herein are combined with conventional therapy for use in the treatment of a disease. In some embodiments, the disease is leukemia or remnant leukemia.

### BRIEF DESCRIPTION OF THE DRAWINGS

FIG. 1 shows NK cell cytotoxicity performed for all patients before therapy and 4 h post-infusion on days 1 and 2. Except for one patient (patient 5), no significant increase in cytotoxicity in the peripheral blood was observed after the administration of the aNK cell infusions.

### DEFINITIONS

In this specification and in the claims that follow, reference will be made to a number of terms that shall be defined to have the following meanings:

As used in the specification and the appended claims, the singular forms “a,” “an” and “the” include plural referents unless the context clearly dictates otherwise.

The term “about” when used before a numerical value is inclusive of the stated value and has the meaning dictated by the context (e.g., includes the degree of error associated with measurement of the particular quantity). The term “about” includes variations that are normally encountered by one of ordinary skill in the art in the field of cancer immunotherapy. For example, the term about indicates that the value can vary by ±0.1, 0.5, 1.0, 2.0, 3.0, 4.0, 5.0, 6.0, 7.0, 8.0, 9.0, or 10.0% of a recited numerical value or range. It is to be understood, although not always explicitly stated, that all numerical designations may be preceded by the term “about.” It is also to be understood, although not always explicitly stated, that the reagents described herein are merely exemplary and that equivalents of such are known in the art.

The term “natural killer (NK) cells” refers to cells of the immune system that kill target cells in the absence of a specific antigenic stimulus, and without restriction according to major histocompatibility complex (MHC) class. Target cells may be tumor cells or cells harboring viruses. NK cells are characterized by the presence of CD56 and the absence of CD3 surface markers.

The term “endogenous NK cells” is used to refer to NK cells derived from a donor (or the patient), as distinguished from the NK-92 cells described herein. Endogenous NK cells are generally heterogeneous populations of cells within which NK cells have been enriched. Endogenous NK cells may be intended for autologous or allogeneic treatment of a patient.

The term “NK-92” refers to natural killer cells derived from the highly potent unique cell line described in Gong et al. (1994), rights to which are owned by NantKwest (hereafter, “NK-92™ cells”). The immortal NK cell line was originally obtained from a patient having non-Hodgkin's lymphoma. Unless indicated otherwise, the term “NK-92™” is intended to refer to the original NK-92 cell lines as well as NK-92 cell lines that have been modified (e.g., by introduction of exogenous genes). NK-92™ cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92™, NK-92™-CD16, NK-92™-CD16-γ, NK-92™-CD16-ζ, NK-92™-CD16(F176V), NK-92™MI, and NK-92™CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK™ cells”). The term “haNK” refers to natural killer cells derived from the highly potent unique cell line described in Gong et al. (1994), rights to which are owned by NantKwest, modified to express CD16 on the cell surface (hereafter, “CD16+ NK-92™ cells” or “haNK® cells”). In some embodiments, the CD16+ NK-92™ cells comprise a high affinity CD16 receptor on the cell surface. The term “taNK” refers tp natural killer cells derived from the highly potent unique cell line described in Gong et al. (1994), rights to which are owned by NantKwest, modified to express a chimeric antigen receptor (hereafter, “CAR-modified NK-92™ cells” or “taNK® cells”). The term “t-haNK” refers to natural killer cells derived from the highly potent unique cell line described in Gong et al. (1994), rights to which are owned by NantkWest, modified to express CD 16 on the cell surface and to express a chimeric antigen receptor (hereafter, “CAR-modified CD16+ NK-92™ cells” or “t-haNK™ cells”). In some embodiments, the t-haNK™ cells express a high affinity CD16 receptor on the cell surface.

The term “recovery” or “recovered” refers to patients that are believed to be free of leukemia by use of conventional therapy but who are statistically at risk of relapse

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between irradiation and infusion should be no longer than four hours in order to preserve optimal activity. Alternatively, NK-92 cells may be inactivated by another mechanism.

As used herein, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

The term “chimeric receptors” as used herein generally refers to an exogenous antibody to a specific antigen on the target cell surface and an activation/stimulation domain.

The term “chimeric antigen receptor” (CAR), as used herein, refers to an extracellular antigen-binding domain that is fused to an intracellular signaling domain of NK-92 cells.

The terms “cytotoxic” and “cytolytic,” when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying the physical integrity of the cell. This results in the killing of the target cell. Without being bound by theory, it is believed that the cytotoxic effect of NK cells is due to cytolysis.

As used herein, “target cells” are the leukemia cells that are killed by the cytotoxic activity of the NK cells described herein.

The terms “leukemia” or “leukemias” refer to malignant neoplasms of the blood-forming tissues. Leukemia includes, but is not limited to, chronic lymphocytic leukemia, chronic myelocytic leukemia, acute lymphoblastic leukemia, acute myelogenous leukemia and acute myeloblastic leukemia. The leukemia can be relapsed, refractory or resistant to conventional therapy.

Leukemias, particularly chronic leukemias, e.g. chronic myelogenous leukemia (CML); chronic myelomonocytic leukemia, and the like, are staged by analysis of the presence of hematopoietic stem and/or progenitor cells, particularly progenitor cells dedicated to the myeloid lineage, which progenitor cells may include CMP (common myeloid progenitors); megakaryocyte erythroid progenitors (MEP), and myelomonocytic lineages (GMP). Staging is useful for prognosis and treatment.

The term “myelodysplastic syndromes” or “a myelodysplastic syndrome” (MDS) as used herein means conditions formerly known as preleukemia that are a diverse collection of hematological medical conditions that involve ineffective production of blood cells. Although frequently asymptomatic, patients with MDS can develop severe anemia, which is treated with blood transfusions. In some cases, the disease worsens and the patient develops cytopenias (low blood counts) caused by progressive bone marrow failure. The outlook in MDS depends on the type and severity of the disease. In one embodiment, NK-92 cells can be used as described herein to treat MDS.

The term “relapse” refers to a situation where patients who have had a remission of leukemia after therapy, followed by a return of leukemia cells in the marrow and a decrease in normal blood cells.

The term “refractory” or “resistant” refers to a circumstance where a patient, even after intensive treatment, has residual leukemia cells in the bone marrow which cells are resistant to such treatment.

The terms “conventional therapy” or “conventional treatment” for leukemia include, but are not limited to, chemotherapy, radiation therapy, hormonal therapy, biological therapy, immunotherapy (other than NK-92 therapy), and the like, and combinations of one or more thereof.

The term “leukemia stem cell,” “cancer stem cell,” or “aberrant stem cell” as used herein refers to a cell that exhibits at least one characteristic of leukemia, and is capable of generating at least one additional, phenotypically distinct cell type. Furthermore, leukemia stem cells are capable of both asymmetric and symmetric replication. It is appreciated that a leukemia stem cell may result from differentiated leukemia cells that acquire stem cell traits and/or stem cells that acquire phenotypes associated with leukemia cells. Common approaches to characterize leukemia stem cells involve evaluation of morphology, cell surface markers, transcriptional profile, and drug response.

As used herein, “endogenous” refers to any material from or produced inside a given organism, cell, tissue or system.

As used herein, “exogenous” refers to any material introduced from or produced outside a given organism, cell, tissue or system.

The term “kill” with respect to a cell or cell population is directed to include any type of manipulation that will lead to the death of that cell or cell population.

The terms “prevent” and “inhibit” are interchangeable and refer to an action that occurs before the patient begins to suffer from a relapse of leukemia. The prevention need not result in a complete prevention of leukemia. Partial prevention, delay of relapse, or reduction of the malignancy is encompassed by this term.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any vertebrate organism including but not limited to mammalian subjects such as humans, domestic animals such as cows, pigs, horses, dogs, cats, rabbits, rats and mice, and non-domesticated animals, or cells thereof whether in vitro or in situ, amenable to the methods described herein.

The term “treatment” or “treating,” to the extent it relates to a leukemia, includes preventing the leukemia from occurring, inhibiting the leukemia, eliminating the leukemia, and/or relieving one or more symptoms of the leukemia, including relapse (e.g. prevents or delays relapse), unless otherwise indicated herein.

The term “therapeutic” as used herein means a treatment and/or prophylaxis. A therapeutic effect is obtained by suppression, remission, or eradication of a disease state.

The term “therapeutically effective amount” includes that amount of NK-92 cells that, when administered, is sufficient to prevent development of, or alleviate to some extent, one or more of the signs or symptoms of leukemia or inhibit its relapse. The therapeutically effective amount of NK-92 cells will vary depending on the leukemia being treated and its severity as well as the age, weight, etc., of the patient to be treated.

Titles or subtitles may be used in the specification for the convenience of a reader, and are not intended to limit the scope of the present disclosure. Additionally, some terms used in this specification are more specifically defined below.

### DETAILED DESCRIPTION OF THE INVENTION

Before the present compositions and methods are disclosed and described, it is to be understood that the aspects described below are not limited to specific compositions, methods, or uses as such may, of course, vary. It is also to be understood that the terminology used herein is for the purpose of describing particular aspects only and is not intended to be limiting.

### NK-92 Cell Line

The NK-92 cell line is a unique cell line that was discovered to proliferate in the presence of interleukin 2 (IL-2). Gong et al., Leukemia 8:652-658 (1994). NK-92 cells are known to have high cytolytic activity against a variety of cancers including leukemia. The NK-92 cell line is a homogeneous cancerous NK cell population having broad anti-tumor cytotoxicity with predictable yield after expansion or proliferation. Phase I clinical trials have confirmed the safety profile of NK-92 cells and anti-leukemia responses in certain patients.

NK-92 cells exhibit the CD56^{bright}, CD2, CD7, CD11a, CD28, CD45, and CD54 surface markers, but do not display the CD1, CD3, CD4, CD5, CD8, CD10, CD14, CD16, CD19, CD20, CD23, and CD34 markers. Growth of NK-92 cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92 has high cytotoxicity even at a low effector:target (E:T) ratio, e.g. 1:1. Gong, et al., supra. NK-92 cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and with 5% carbon dioxide. Koepsell, et al., Transfusion 53:398-403 (2013). However, endogenous NK cells are significantly different from NK-92 cells, in large part because of their distinct origins: NK-92 is a cancer-derived cell line, whereas endogenous NK cells are harvested from a donor (or from the patient of interest) and processed for infusion into the patient. Endogenous NK cell preparations are heterogeneous cell populations, whereas NK-92 cells are a homogeneous, clonal cell line. NK-92 cells readily proliferate in culture while maintaining cytotoxicity, whereas endogenous NK cells do not. In addition, a heterogeneous population of endogenous NK cells, unlike NK-92 cells, does not aggregate at high density.

In some embodiments, NK-92 cells comprise a medium, such as human serum or an equivalent thereof. In some embodiments, the medium comprises human serum albumin. In some embodiments, the medium comprises human plasma. In some embodiments, the medium comprises about 1% to about 15% human serum or human serum equivalent. In some embodiments, the medium comprises about 1% to about 10% human serum or human serum equivalent. In some embodiments, the medium comprises about 1% to about 5% human serum or human serum equivalent. In a preferred embodiment, the medium comprises about 2.5% human serum or human serum equivalent. In some embodiments, the serum is human AB serum. In some embodiments, a serum substitute that is known in the art and acceptable for use in human therapeutics is used instead of human serum. Although concentrations of human serum over about 15% can be used, it is contemplated that concentrations greater than about 5% could be cost-prohibitive.

In various embodiments, the NK-92 cells administered to a patient include original NK-92 cells as described herein, as well as genetically modified NK-92 cells, such as original NK-92 cells modified to express CD16 or any marker disclosed herein. Exemplary NK-92 cells include, but are not limited to, NK-92 cell lines available from American Type Culture Collection (ATCC) under Accession Nos.: PTA 6670, PTA 6672, PTA 8836, PTA 8837, CRL-2407 and CRL-2408.

In other embodiments, the NK-92 cells administered to a patient include NK-92 cells modified to express chimeric antigen receptors (CARs). Methods for engineering NK-92 cells to express CARs are described in Boissel et al., Oncoimmunology 2: 10, e26527 (2013), which is hereby incorporated by reference in its entirety.

### Methods of Treatment

Provided herein are methods of using NK-92 cells for treating patients having leukemia or patients that are genetically pre-disposed to leukemia. In one embodiment, such methods include treatment of a patient in recovery from leukemia. It is to be understood that, without being limited by a particular theory, when such NK-92 cells are introduced into patients, the cells eradicate residual and/or recalcitrant leukemia cells, including leukemia stem cells, or the NK-92 cells can be used as primary therapy after relapse of the leukemia wherein the leukemia prior to relapse was treated with conventional therapy.

As noted above, the methods described herein are directed to treating leukemia, including, but not limited to, acute T-cell leukemia, acute myelogenous leukemia (AML), acute promyelocyte leukemia, acute myeloblasts leukemia, acute megakaryoblastic leukemia, precursor B acute lymphoblastic leukemia, precursor T acute lymphoblastic leukemia, Burkett's leukemia, or acute biphenotypic leukemia; a chronic leukemia, e.g., chronic myeloid lymphoma, chronic myelogenous leukemia (CML), chronic monocytic leukemia, chronic lymphocytic leukemia (CLL), or B-cell prolymphocytic leukemia; T-cell prolymphocytic leukemia, as well as patients that are genetically pre-disposed to leukemia.

The present disclosure encompasses methods of treating patients who have been previously treated for leukemia, but who retain or are suspected of retaining leukemia cells which are recalcitrant to standard therapies. The disclosure also encompasses methods of treating patients regardless of patient's age, although some leukemias are more common in certain age groups. Since patients with leukemia have heterogeneous clinical manifestations and varying clinical outcomes, the treatment given to a patient may vary, depending on his/her prognosis, all of which are within the purview of the skilled clinician.

Patients suitable for treatment by these methods include individuals who have previously been treated for leukemia and that are in recovery (e.g., remission). Other patients suitable for the methods described herein are those who are considered at high risk for experiencing a relapse of leukemia following conventional treatment(s). Treatment regimens include the eradication of leukemia cells by administering NK-92 cells to a patient. Accordingly, methods encompassed by the present disclosure comprise administering one or more doses of NK-92 cells to such patients.

In certain embodiments, an effective amount of NK-92 cells is administered to such patients, in any amount or number that results in a detectable therapeutic benefit or manifestation to the individual. A detectable therapeutic benefit is where, for example, patients are evaluated for blast clearance in the bone marrow to less than 5% of all nucleated cells, morphologically normal haematopoiesis, and return of peripheral blood cell counts to normal levels. In some embodiments, an absolute number of NK-92 cells can be administered to such a patient, e.g., at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells. In other embodiments, NK-92 cells can be administered to such a patient by relative numbers of cells, e.g., at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the patient. In other embodiments, the total dose may calculated by m2 of body surface area, including about 1×10¹¹, 1×10¹⁰, 1×10⁹, 1×10⁸, or 1×10⁷, per m². The average person is about 1.6 to about 1.8 m².

NK-92 cells can also be administered to such a patient according to an approximate ratio between a number of NK-92 cells and a number of suspected leukemia cells in the patient. For example, NK-92 cells can be administered to the patient at a ratio of about, at least about or at most about 1:1, 1:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1 to the estimated number of leukemia cells in the individual. The number of leukemia cells in such a patient can be estimated, e.g., by counting the number of leukemia cells in a sample of tissue from the patient, e.g., blood sample, biopsy, or the like.

The NK-92 cells, and optionally other anti-leukemia agents, can be administered one time to a patient having relapsed leukemia or can be administered to a patient multiple times, e.g., once every 1 , 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy. In embodiments in which NK-92 cells and an immunomodulatory compound or thalidomide are used, the immunomodulatory compound or thalidomide, and cells or perfusate, can be administered to the individual together, e.g., in the same formulation; separately, e.g., in separate formulations, at approximately the same time; or can be administered separately, e.g., on different dosing schedules or at different times of the day. The perfusate, perfusate cells, natural killer cells, e.g., PINK cells, pools and/or combinations of the same can be administered without regard to whether perfusate, perfusate cells, natural killer cells, e.g., PINK cells, pools and/or combinations of the same have been administered to the individual in the past.

In one aspect, a method is provided for killing residual, or remnant, leukemia cells in a patient, wherein the patient is recovering from a treatment for leukemia, in which the method comprises administering to the patient one or more doses of NK-92 cells sufficient to kill all, or substantially all, of the remnant leukemia cells remaining in the patient. In various embodiments, secondary therapies involve the administration of NK-92 cells to a patient after the patient has undergone treatment under conventional therapy, wherein the administration of NK-92 cells can prevent the maintenance and/or development of remnant leukemia cells, including aberrant and recalcitrant leukemia stem cells.

For example, prior to administering the NK-92 cells to the patient, the remnant leukemia cells may be present in the patient at a level that is less than about 20%, less than about 10%, less than about 5% or less than about 1% of the level of leukemia cells that was detected in the patient prior to the treatment for leukemia.

In some embodiments, the remnant leukemia cells comprise leukemic stem cells. The remnant leukemia cells may also include marrow cell precursors of lymphocytes, red blood cells, white blood cells or platelets.

In some embodiments, the treatment for leukemia includes conventional therapy, such as chemotherapy, radiotherapy, hormone treatment or a bone marrow transplant, and said remnant leukemia cells were and remain substantially resistant to the conventional therapy.

### Methods of Treatment to Inhibit Relapse

In another aspect, a method is provided for inhibiting a relapse of leukemia in a patient, wherein the patient is recovering from a treatment for leukemia, in which the method comprises administering to the patient one or more doses of NK-92 cells sufficient to inhibit a relapse of leukemia in the patient. In an embodiment, NK-92 cells can be administered after a patient undergoes treatment under conventional therapies, such as chemotherapy, where administration of NK-92 cells can prevent recurrence of leukemia, i.e., relapse.

In some embodiments, the relapse of leukemia in the patient is inhibited for at least about one, at least about two, at least about three, at least about four, at least about six, or at least about twelve months following the administration of the NK-92 cells.

In some embodiments, the treatment for leukemia comprises conventional therapy, such as one or more of chemotherapy, radiotherapy, hormone treatment, bone marrow transplant, biological therapy, or immunotherapy (other than NK-92 therapy).

### Methods of Treatment After Relapse

In another aspect, a method is provided for treating leukemia following relapse of leukemia in a patient, wherein the patient experiences a relapse of leukemia following treatment with conventional therapies. In some embodiments, the method comprises administering to the patient one or more doses of NK-92 cells sufficient to result in a therapeutic benefit to the patient.

In some embodiments, NK-92 cells can be used in combination with another agent or therapy method to treat a patient who has experienced relapse of leukemia following conventional treatment. Examples of anti-leukemic agents include, but are not limited to, mda-7, human fibroblast interferon, mezerein, and Narcissus alkaloid (pretazettine). The combined use of NK-92 cells and conventional anti-leukemic agents may provide a unique treatment regimen that is unexpectedly effective in certain patients. Without being limited by theory, it is believed that NK-92 cells may provide additive or synergistic effects when given concurrently with conventional anti-leukemic agents to a patient who has experienced relapse of leukemia following conventional therapy.

### Pharmaceutical Compositions

In another aspect, provided are pharmaceutical compositions comprising a therapeutic dose of NK-92 cells for the treatment of leukemia following treatment with conventional therapies.

NK-92 pharmaceutical compositions may be administered in a manner as determined to be appropriate by a qualified clinician (e.g., iv administration). The quantity and frequency of administration will be determined by such factors as the condition of the patient, and the type and severity of the patient's disease, although appropriate dosages may be determined by clinical trials.

When “an effective amount” is indicated, the desired amount of NK-92 cells to be administered can be determined by a clinician with consideration of individual differences in age, weight, tumor size, extent of infection or metastasis, and condition of the patient. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In a further embodiment, the cell compositions described herein are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions described herein are administered following B-cell ablative therapy, such as agents that react with CD20, e.g., Rituxan. For example, in one embodiment, patients may undergo standard treatment with high dose chemotherapy followed by peripheral blood stem cell transplantation. In certain embodiments, following the transplant, patients receive an infusion of NK-92 cells described herein.

The following examples are included to illustrate and not to limit the claimed subject matter. All publications or references cited in the present specification are hereby incorporated by reference in their entireties.

### EXAMPLES

### Example 1

This Example provides the results from a phase 1 clinical trial with adoptively transferred aNK cells in patients with refractory and relapsed AML. The objectives were to determine safety and feasibility of this adoptive cell therapy in pretreated AML patients and to investigate effects of aNK cell infusions on the patient's immune system. The results demonstrate the safety and feasibility of adoptive cell therapy with “off-the-shelf”' aNK cells in patients with refractory/relapsed AML.

### Methods

### Patients

Patients aged 18 years or older with relapsed/refractory AML, as defined by the World Health Organization classification [Swerdlow S H, International Agency for Research on Cancer, World Health Organization. WHO classification of tumours of haematopoietic and lymphoid tissues. International Agency for Research on Cancer; 2008], were eligible for this trial and were treated at the University of Pittsburgh after providing written informed consent. Other key eligibility criteria were Eastern Cooperative Oncology Group performance status ≤2 and adequate end-organ function. Exclusion criteria included a diagnosis of acute promyelocytic leukemia, symptomatic central nervous system involvement, left ventricular ejection fraction <45% and prior history of allogeneic hematopoietic cell transplantation. Neutropenia, anemia and thrombocytopenia were not exclusion criteria as these abnormalities were expected in patients with relapsed/refractory AML. The protocol was reviewed by the University of Pittsburgh Institutional Review Board and approved according to institutional guidelines (clinicaltrials.gov identifier NCT00900809).

### Clinical Grade aNK Cells

Clinical grade aNK cells were expanded in the GMP facility of the Center for Cell and Gene Therapy at the Baylor College of Medicine, Houston, Tex., as a project supported by the Production Assistance for Cell Therapies (NHLBI-PACT) program. A vial of cells containing 20×10⁶ cells was provided by NantKwest Inc, from the cryopreserved working cell bank. The cells were thawed and cultured in X-VIVO 10 medium without gentamicin or phenol red (Lonza), supplemented with 5% (v/v) human AB serum (Valley Biomedical), 450 IU/mL IL-2 (USP grade Proleukin; Novartis Vaccines and Diagnostics), 0.036 mmol/L asparagine (Sigma-Aldrich), 0.45 mmol/L L-glutamine (Gibco Thermo Fisher Scientific) and 0.32 mmol/L L-serine (Sigma-Aldrich). For the first 10 days, cells were cultured in T-25 flasks, and then 5-10×10⁶ aNK cells were transferred into G-Rex10 units (Wilson Wolf Manufacturing Corporation) with 10-40 mL of culture medium. When cell numbers exceeded 50×10⁶, the cells were transferred to other G-Rex100 flasks, and culture was continued to obtain 2-8×10⁵ cells/mL. Freshly harvested aNK cells were shipped overnight to the University of Pittsburgh at the cell concentration of 1-5×10⁶/mL in complete culture medium in several G-Rex100 vessels with 37° C. pre-warmed gel packs.

Upon arrival at the Immune Monitoring and Cell Products Laboratory at the University of Pittsburgh, an aliquot consisting of half of the shipped cells was washed in X-VIVO 10 medium (without supplements), irradiated at 1000 cGy as required by the U.S. Food and Drug Administration to prevent further cell proliferation and resuspended in 5% (v/v) human serum albumin (Buminate, Baxter). The irradiation dose did not reduce cytotoxicity of aNK cells. The aliquot containing the other half of cells was transferred to an incubator at 37° C., in the atmosphere of 5% CO₂ in air without any manipulation and incubated over-night. These cells were prepared for the second cell infusion as described earlier. The release criteria for aNK cells were >90% CD56⁺CD3⁻ cells; <5% CD16⁺CD3⁺ cells, >70% cell viability, negative for bacteria by the BACTEC assay and Gram stain, negative for myco-plasma by MycoAlert and ≤5 EU/dose of endotoxin by Kinetic-QCL.

### Study Design and Treatment Plan

This phase 1 clinical trial was a single-center, open-label, dose escalation study. Two cell dose levels were used: 1×10⁹ cells/m² and 3×10⁹ cells/m². These doses were chosen on the basis of prior clinical experience using aNK cells in patients with solid tumors [Arai S et al., Cytotherapy 2008; 10(6):625-32; Tonn T et al., Cytotherapy 2013; 15(12):1563-70]. Patients were enrolled to dose levels based on the traditional 3+3 dose-escalation design. Patients were enrolled sequentially with their dose determined by their enrollment sequence. No intra-patient dose escalation was allowed.

The administration of the aNK cells was performed in the outpatient setting. One treatment course consisted of two infusions of the same cell dose, each cell infusion administered 24 h apart (days 1 and 2). Patients were pre-medicated 15 min before the aNK, cell infusion with diphenhydramine (25 mg) administered intravenously and acetaminophen (500 mg) administered orally. aNK cells were administered intravenously over 60 min, and patients were monitored for 4 h post-infusion. The same procedure was followed for the second infusion of aNK cells given on day 2. This second infusion was only administered if no dose-limiting toxicities attributable to the first infusion of aNK were encountered. Patients under-went bone marrow biopsy 21 days after each course of therapy. Patients who had stable disease or reduction of leukemia blasts were eligible to receive additional aNK infusions.

### Clinical Toxicity and Response Evaluation

Adverse events were characterized in terms of attribution and severity and are reported according to the NCI Common Terminology Criteria for Adverse Events (CTCAE) v4.0. Disease assessment was performed 21 days after aNK cell administration and the bone marrow biopsy was evaluated for response using established criteria [Cheson B D et al., J Clin Oncol 2003; 21(24):4642-9].

### Immunophenotyping NK Cell Cytotoxicity and Cytokines

Samples of venous blood (20-50 mL) were obtained before each aNK cell infusion, 4 h after each infusion and on days 4,7 and 21 post-infusion. The blood was drawn into heparinized tubes, hand-carried to the laboratory and immediately processed using Ficoll-Paque Plus (GE Healthcare) gradients. The collected blood was used to measure levels of plasma cytokines, NK cell activity and for flow cytometric analysis. The supplementary material describes the laboratory methods used.

### Statistical Analysis

All patients who received aNK cells were included in the safety analysis. Safety data were summarized by frequency and severity of adverse events using descriptive statistics. For each patient and each cytokine, differences between each time point and baseline were calculated. On the basis of the differences, we tested whether the two cell doses had the same effect using two-sided two-sample t-test and two-sided one-sample t-test. For the cytotoxicity assays and flow cytometry, two-sided paired t-test was used to examine changes in the activity, receptor expression and immune cells subsets from baseline and each time point. The data were presented as means±SE or SD at each time of measurement. P values of <0.05 were considered statistically significant. Statistical analysis was performed using SAS software.

### Results

### aNK Cell Culture, Phenotype and Cytotoxicity

The median expansion of aNK cells in the GMP facility at the Baylor College of Medicine, Houston, Tex., was 24 days (range 20-28 days). Overnight shipments of aNK cells to the University of Pittsburgh were executed efficiently with no delays in the arrival of cells. On arrival, the cells were transferred from the G-Rex100 vessels into the final infusion bag and were evaluated for sterility, viability and phenotype. All cell products met the release criteria.

The mean cell aNK cell viability on day 1 was 96% range 95%-98%) and 94% (range 89%-94%) on day 2. The mean aNK cell cytotoxicity for all products before infusion was 4409±2606 lytic units (LU) on day 1 and 3628±2064 LU on day 2. The phenotype of aNK cells was CD3⁻, CD56⁺, CD16⁻, NKG2D⁺, CD94⁺, NKG2A⁺, CD158a⁻ and CD158b⁻.

### Patient Characteristics and Treatment

Seven patients with refractory or relapsed AML enrolled into the study were treated with a total of 20 aNK cell infusions. The median age was 71 years (range 56-80 years), and all patients had previously received multiple therapies for AML. Patient demographics, base-line characteristics and prior therapies are presented in Error! Reference source not found. The first three patients were treated with the cell dose of 1×10⁹ cells/m² and four patients with the cell dose of 3×10⁹ cells/m² (Table II).

### Safety

None of the seven patients experienced dose-limiting toxicity (DLT) during the aNK cell administration or during the 21 days observation period post-infusion. No grade 3-4 toxicities related (probable or definite) to the aNK cell infusion occurred. One patient developed grade 2 fever and chills following each aNK cell infusion that required hospitalization; these known effects were reversible with supportive care, intravenous hydration and antipyretics. Hospitalizations that occurred during the observation period were not related to the aNK cell infusions and included central-line related bacteremia, neutropenic fever, red blood cell and platelet transfusion and pneumonia.

### Immune Cells in the Peripheral Blood

No significant changes in the percentage and absolute numbers of CD4⁺, CD8⁺ T cells, T regulatory cells, NK cell subsets or myeloid-derived suppressor cells (MDSC) were observed at 4 h after each infusion and on days 7 and 21 (Table III), To distinguish aNK cells from endogenous NK cells after adoptive transfer by flow cytometry, gates were set on CD3⁻, CD56⁺, CD16⁻, CD158a⁻, CD158a⁻ cells and expression levels of NK-cell receptors was determined. No significant changes in expression levels of the natural cytotoxicity receptors NKp30, NKp44, NKp46, NKG2D and NKG2A were observed (data not shown).

### NK Receptor/Ligands and NK Cell Activity

Because aNK-cell activity depends on receptor-ligand interactions, we evaluated the expression levels of NK cell ligands on leukemia blasts. Using flow cytometry, leukemia blasts were gated based on the expression of CD45, CD33, CD34 and CD117. The percentage of blasts expressing ULPB1, ULPB2 and MICA/B was 4.3±5.2, 1.6±1.5 and 9.2±16, respectively. No significant changes in the percentage of blasts expressing these ligands were observed 4 h after each aNK infusion and on days 7 and 21.

TABLE I
Patients’ baseline characteristics.

| | | | | | | % blasts in | % blasts | WBC | | |
|---|---|---|---|---|---|---|---|---|---|---|
| | Age | | ECOG | | Cytogenetic and | peripheral | in bone | count | Hg | Platelet |
| Pt | (y) | Sex | PS | Prior AML therapies | molecular studies | blood | marrow | (10⁹/L) | (g/dL) | count |
| 1 | 59 | M | 1 | idarubicin + cytarabine → | 46 XY Negative for FLT3- | 1 | 12 | 1.8 | 10.9 | 70 |
| | | | | idarubicin + cytarabine → | ITD mutation, negative | | | | | |
| | | | | high dose cytarabine → | for NPM1 mutation | | | | | |
| | | | | dasatinib + all-trans | | | | | | |
| | | | | retinoic acid | | | | | | |
| 2 | 56 | F | 2 | idarubicin + cytarabine → | del (5) (q21q31), −7, dic | 1 | 70 | 0.1 | 9.6 | 19 |
| | | | | idarubicin + cytarabine → | (21; 22)(q22; p11.2) | | | | | |
| | | | | fludarabine + cytarabine → | | | | | | |
| | | | | mitoxantrone + etoposide → | | | | | | |
| | | | | clofarabine + cytarabine | | | | | | |
| 3 | 58 | M | 1 | idarubicin + cytarabine → | −5, t(7; 18) (p10; q10)del | 33 | 15 | 0.8 | 8.5 | 19 |
| | | | | mitoxantrone + etoposide → | (7), (q22q36), dic(17; 20) | | | | | |
| | | | | fludarabine + cytarabine | (p12; q11.2), −18, −21 | | | | | |
| 4 | 71 | M | 2 | idarubicin + cytarabine → | del(12) (p11.2p13), +8, +21 | 21 | 32 | 3.8 | 9.4 | 18 |
| | | | | cytarabine + decitabine | | | | | | |
| 5 | 78 | M | 1 | Cytarabine + decitabine → | 46 XY Negative for FLT3- | 1 | 33 | 1.0 | 8.7 | 16 |
| | | | | decitabine → azacitidine | ITD mutation, positive | | | | | |
| | | | | | for NPM1 mutation | | | | | |
| 6 | 76 | M | 1 | Cytarabine + decitabine | 46 XY | 14 | 56 | 3.3 | 10 | 38 |
| 7 | 80 | M | 1 | Cytarabine + decitabine → | 46 XY | 1 | 46 | 1.6 | 9.5 | 66 |
| | | | | decitabine | | | | | | |

ECOG, Eastern Cooperative Oncology Group; F, female; M, male; FLT3, FMS-like tyrosine kinase-3; Hg, hemoglobin; NPM1, nucleophosmin; PS, performance status; Pt, patient; WBC, white blood cell.

TABLE II
aNK cell therapy and treatment outcomes.

| | | Day 1-2 | | | Total number | |
|---|---|---|---|---|---|---|
| | | infusion | Dose-limiting | Number | of aNK | |
| Pt | aNK cell dose | reaction | toxicities | of courses^{a} | infusions | Outcome |
| 1 | 1 × 10⁹/m² | — | — | 1 | 2 | Progressive leukemia, ↑ BM |
| | | | | | | blasts 12%-39% |
| 2 | 1 × 10⁹/m² | — | — | 2^{b} | 4 | ↓ BM blasts 70%-48% after |
| | | | | | | first course, retreated ×1 |
| | | | | | | course → progressive |
| | | | | | | leukemia,↑WBC |
| | | | | | | (49 × 10⁹/L) |
| | | | | | | and % peripheral blasts (36%) |
| 3 | 1 × 10⁹/m² | — | — | 3^{c} | 6 | Stable % BM blasts (15%) after |
| | | | | | | first course, retreated ×2 |
| | | | | | | courses with stable % BM |
| | | | | | | blasts → progressive |
| | | | | | | leukemia, ↑ BM blasts to 28% |
| 4 | 3 × 10⁹/m² | — | — | 1 | 2 | Not evaluable, developed |
| | | | | | | pneumonia-ceased to breath |
| | | | | | | 14 days post-infusion due |
| | | | | | | to pneumonia |
| 5 | 3 × 10⁹/m² | — | — | 1 | 2 | Progressive leukemia, ↑ BM |
| | | | | | | blasts 33% to 95% |
| 6 | 3 × 10⁹/m² | — | — | 1 | 2 | Progressive leukemia, ↑ WBC |
| | | | | | | (45 × 10⁹/L) and % |
| | | | | | | peripheral blasts (45%) |
| 7 | 3 × 10⁹/m² | Grade 2 fever | — | 1 | 2 | Stable % BM blasts (44%) after |
| | | and rigors | | | | first course, no further |
| | | | | | | treatment due to experienced |
| | | | | | | infusion reaction |

BM, bone marrow; Pt, patient.
^{a}A course consisted of two aNK cell infusions, administered 24 h apart.
^{b}Thirteen days required for the expansion of the aNK cells for the second course of therapy.
^{c}Nineteen days and 5 days required for the expansion of the aNK cells for the second and third course of therapy, respectively.

TABLE III
Lymphocyte subsets prior and after aNK cell therapy.

| | Day 1 | Day 1 | Day 2 | Day 2 | Day 7 | Day 21 |
|---|---|---|---|---|---|---|
| | pre-therapy | post-therapy | pre-therapy | post-therapy | post-therapy | post-therapy |
| | mean (%) ± SD | mean (%) ± SD | mean (%) ± SD | mean (%) ± SD | mean (%) ± SD | mean (%) ± SD |
| Immune cell subsets | | | | | | |
| CD3⁺ T cells | 27.4 ± 26 | 26.2 ± 29 | 29.2 ± 30 | 17.8 ± 17.5 | 27.5 ± 28.2 | 18.6 ± 27.2 |
| CD8⁺ | 4.8 ± 3.5 | 3.1 ± 2.6 | 5.1 ± 4.5 | 3.1 ± 2.6 | 5.2 ± 5.6 | 5.1 ± 7.1 |
| CD8⁺, CD69⁺ | 4.4 ± 2.6 | 4.1 ± 3.3 | 5.3 ± 4.7 | 6.7 ± 6.3 | 3.1 ± 2.2 | 4.4 ± 3.6 |
| CD4⁺ | 20.7 ± 2 | 21.0 ± 27.7 | 22.6 ± 27.3 | 12.9 ± 12.8 | 20.3 ± 21.8 | 11.3 ± 16.7 |
| CD4⁺, CD69⁺ | 1.0 ± 0.9 | 0.7 ± 0.4 | 0.9 ± 0.5 | 0.9 ± 0.3 | 0.8 ± 0.3 | 2.0 ± 2.7 |
| Treg | | | | | | |
| CD4⁺, CD25hi⁺ | 1.5 ± 0.5 | 1.5 ± 0.9 | 1.6 ± 1.1 | 1.1 ± 1.0 | 0.9 ± 0.5 | 0.9 ± 0.9 |
| CD4⁺, CD25hi⁺, Foxp3⁺ | 75.9 ± 17.1 | 76.8 ± 16.7 | 73.3 ± 19.5 | 67.5 ± 22.5 | 80.4 ± 18.9 | 66.6 ± 14.6 |
| CD4⁺, CD25hi⁺, CD39⁺ | 54.0 ± 23.5 | 53.4 ± 26.9 | 54.7 ± 24.0 | 54.7 ± 27.7 | 56.9 ± 31.9 | 43.3 ± 28.9 |
| NK cells | | | | | | |
| CD3⁻, CD16⁺, CD56⁺ | 13.5 ± 20.3 | 7.3 ± 9.2 | 11.2 ± 9.6 | 5.5 ± 7.2 | 15.8 ± 12.9 | 13.5 ± 19.5 |
| CD3⁻, CD16−, CD56⁺ | 16.7 ± 25.2 | 13.8 ± 21.6 | 14.9 ± 23.0 | 16.7 ± 24.3 | 18.3 ± 26.7 | 19.3 ± 27.0 |
| CD3−, CD16+, CD56⁺, NKG2D⁺ | 79.1 ± 14.0 | 73.8 ± 19.8 | 80.3 ± 16.8 | 79.6 ± 17.8 | 84.8 ± 9.6 | 72.6 ± 19.0 |
| CD3⁻, CD16⁻, CD56+, NKG2D+ | 35.7 ± 29.1 | 33.1 ± 28.6 | 34.3 ± 33.2 | 26.0 ± 28.2 | 33.8 ± 35.4 | 21.4 ± 22.7 |
| MDSC Lineage negative | | | | | | |
| (CD3⁻CD56⁻CD19⁻), | 3.1 ± 3.5 | 3.8 ± 3.1 | 2.4 ± 2.2 | 2.2 ± 2.9 | 3.3 ± 2.8 | 0.9 ± 1.0 |
| DR⁻CD33⁺CD11b⁺ | | | | | | |

Lymphocyte subsets were measured before aNK therapy, 4 h after each infusion and on days 7 and 21 post-infusion. No significant change in the percentage of lymphocyte subsets monitored was detected. Treg, T regulatory cells; MDSC, myeloid-derived suppressor cells.

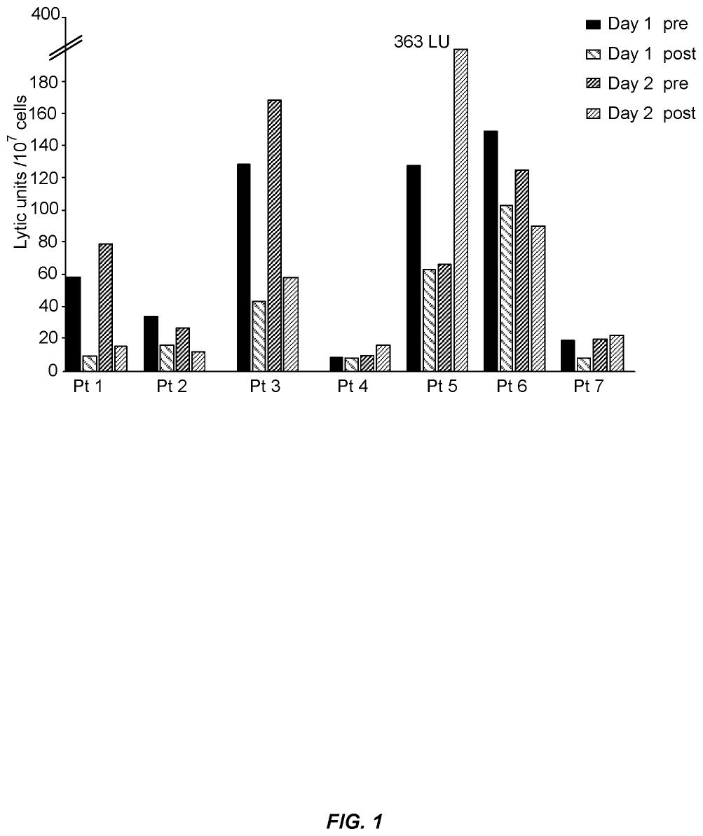

Post-infusion mean NK-cell cytotoxicity measured 4 h after the first adoptive transfer in patients' blood decreased in all patients compared with pre-treatment cytotoxicity levels (74±59 LU pre-therapy versus 34.8±37 LU post-therapy, P<0.01) and then recovered to pre-treatment levels within 24 h (69.7±59 LU) and did not change following the second aNK cell infusion (82.2±128 LU) except in patient 5, in whom NK cell cytotoxicity increased from 65 to 364 LU after the second aNK cell infusion (FIG. 1). Overall, no significant increase in cytotoxicity of circulating NK cells was observed after the administration of the aNK cell infusions.

### Plasma Cytokines

A significant reduction in the plasma levels of fibroblast growth factor (P=0.0285), granulocyte colony-stimulating factor (P=0.0345) and RANTES (P=0.0486) were observed at 24 h after therapy (see supplementary material). An increase in the level of IL-1Rα (P=0.0243) occurred 4 days after therapy with both aNK cell doses (1×10⁹ cells/m² and 3×10⁹ cells/m²). A dose-dependent effect in the levels of IL-6, IL-1RA, IL-10, vascular endothelial growth factor (VEGF) and IP-10 was observed; while the levels of these cytokines did not change after infusion of the lower dose of aNK cells, with the higher aNK cell dose, the levels of IL-6 (P=0.0293) and IL-1Rα (P=0.0231) increased on day 7 after treatment, and the levels of IL-10 (P=0.0252), VEGF (P=0.0478), and IP-10 (P=0.0478) were increased 21 days after treatment. No significant changes on the levels of IL-15, an NK cell homeostatic cytokine, were observed.

### Efficacy

All seven patients completed the first course of therapy; six patients were evaluable for response 21 days after therapy (Table II). No patient achieved complete remission. In one patient, the blast percentage was reduced from 70% to 48% after a course of therapy, and the patient received an additional course of aNK cells. In two patients, the blast percentage remained stable after the first course of therapy; one of these two patients received additional two courses of therapy with aNK cells.

### Discussion

Clinical use of NK cells is an area of intense investigation. Ex vivo activated NK cells have been used for therapy of hematological or solid cancers because NK cells that lyse tumor cells provide the first line of anti-tumor defense [Knorr D A, Bachanova V, Verneris M R, Miller J S. et al., Semin Immunol 2014; 26(2):161-72; Miller J S., Hematology 2013; 2013:247-53]. aNK cells mediate high levels of cytotoxic activity against a broad spectrum of primary and cultured tumor cells, including AML blasts [Klingemann Boissel L, Toneguzzo F., Front Immunol 2016; 7:91; Suck G et al., Cancer Immunol Immunother 2016; 65(4):485-92] and have a well-characterized and stable immune phenotype that favors therapeutic utility. They express activating receptors but lack most of the inhibitory KIRs, and thus retain cytotoxicity against cancer cells that express major histocompatibility complex class I molecules. They can be cultured under current GMP conditions to yield large numbers of uniformly potent effector cells required for adoptive transfer in a clinical setting. They represent an “off-the-shelf product that can be shipped for therapy to a distant location and safely administered to patients with advanced malignancies as shown in this report. Some patients with solid cancers who received therapy with aNK cells achieved clinically significant responses [Arai S et al., Cytotherapy 2008; 10(6):625-32; Tonn T et al., Cytotherapy 2013; 15(12):1563-70]. These positive earlier reports in patients with solid cancers and the anti-leukemia effects of aNK cells motivated us to initiate first ever adoptive therapy of aNK cells for patients with refractory/relapsed AML.

Patients with relapsed/refractory AML could benefit from transfer of allogeneic aNK cells to replace dysfunctional autologous NK cells and to restore, at least in part, anti-leukemia activity. Allogeneic NK cells have been previously evaluated for safety and efficacy in AML patients both in the transplant and non-transplant settings. NK-cell alloreactivity based on KIR epitope mismatches in AML, patients undergoing haploidentical hematopoietic cell transplantation (HCT) demonstrated improved survival rates [Ruggeri L et al., Science 2002; 295(5562):2097-100; Ruggeri L et al., Blood 2007; 110(1):433-40]. Adoptive therapy using haploidentical NK cells in combination with chemotherapy to lympho-deplete the recipient and facilitate expansion of the allogeneic NK cells with IL-2 administration induced CR in patients with relapsed and refractor AML [Bachanova V et al., Blood 2014; 123(25):3855-63; Curti A et al., Blood 2011; 118(12):3273-9; Miller J S. et al., Blood 2005; 105(8):3051-7; Rubnitz J E et al., J Clin Oncol 2010; 28(6):955-9].

For AML patients who are not candidates for allogeneic HCT and are refractory to chemotherapy, treatment options are limited. As aNK cells mediate robust anti-leukemia activity, their transfer could be beneficial for these relapsed/refractory AML patients, provided it can be tolerated with no toxicities. The phase 1 trial we conducted showed that aNK cell delivery was safe. No dose-limiting toxicity occurred in patients with relapsed/refractory AML who received a total of 20 aNK, cell infusions. One patient developed infusion related toxicity that was reversible with supportive care.

In considering adoptive aNK cell therapy, it is necessary to consider the use of a lympho-depleting preparative regimen to promote NK cell expansion and strategies to enhance NK cytotoxicity post-infusion such as the use of cytokines or depletion of T regulatory cells [Bachanova V et al., Blood 2014; 123(25):3855-63; Miller J S. et al., Blood 2005; 105(8):3051-7]. In the current study, no such strategy was used as aNK, cells were transferred to a profoundly immunosuppressive environment already compromised by previous chemotherapies, the aNK cells did not proliferate post-infusion due to the irradiation used before each infusion and the safety and toxicity of the aNK cells were not compounding by additional therapies before or after transfer of the aNK cells.

We expected that delivery of aNK cells would result in at least partial immune cell reconstitution in AML patients due to the reduction of leukemia blasts and/or changes in the host cytokine milieu induced by transferred aNK cells. Therefore, the patients' immune status was monitored immediately before and at various times after therapy. As expected, the heavily pretreated AML patients monitored before therapy were immunocompromised with low lymphocyte counts, including a low frequency of NK cells, and depressed anti-leukemia cytotoxicity of NK cells in the peripheral blood. After adoptive therapy, no significant change in the absolute number or percentage of immune cells was observed. The phenotypic profile of immune cells did not change, and, with one exception, no increases in cytotoxicity of the NK cells in the circulation were observed after therapy. Instead, at 4 h after the aNK cell infusions, a significant drop in cytotoxicity levels of NK cells in the peripheral blood was observed, presumably due to NK cell migration to the lungs and liver [Nannmark U et al., In Vivo 2000; 14(5):651-8; Pegram H J et al., Cancer Immunol Immunother 2010; 59(8):1235-46]. However, it is equally likely that circulating immunosuppressive factors contributed to a loss of function in adoptively transferred aNK cells and homing mechanisms of the aNK cells in the bone marrow [Boyiadzis M et al., Blood 2016; 128:1609].

The pre-therapy blood cytokine profile in the AML patients was highly variable, with highly elevated levels of IL-1β, IL-1Rα, MCP-1, and IL-12 in some patients. Plasma levels of several cytokines (granulocyte colony-stimulating factor, fibroblast growth factor and RANTES) decreased at 24 h after therapy, and that of IL-1Rα increased on days 4 and 7 post-therapy. A cell-dose effect on levels of several cytokines in the blood was observed, with significant increases in levels of pro-inflammatory IL-1Rα and IL-6 on day 7 and in levels of immunosuppressive IL-10, IP-10 and VEGF on day 21 only post infusion of the high aNK-cell dose.

Interestingly we did not detect significant changes in the IL-15 levels, a cytokine that plays a major role in NK-cell differentiation, expansion in the periphery and survival. Plasma IL-15 levels have been demonstrated to increase significantly following cytoreductive therapy [Miller J S. et al., Blood 2005; 105(8):3051-7; Boyiadzis M et al., Biol Blood Marrow Transplant 2008; 14(3):290-300; Chik K W et al., J Pediatr Hematol Oncol 2003; 25(12):960-4]. The reported elevated plasma IL-15 levels could be related to the regimen-induced depletion of lymphoid populations that normally consume circulating IL-15. This may explain our IL-15 data because no lympho-depleting preparative regimen was used in the current study.

In aggregate, these data suggest that adoptive aNK cell therapy had transient but measurable effects on the cytokine profile initially supporting and later deflating inflammatory responses. It is thus likely that aNK cell doses, numbers of infusions and the patient's compromised immune system may have contributed to the observed lack of immune restoration after therapy.

In conclusion, the trial demonstrated the safety and feasibility of adoptive cell therapy with “off-the-shelf” aNK cells in patients with refractory/relapsed AML. No grade 3-4 toxicity occurred with the maximal cell dose used. These data provide the foundation for future combination immunotherapy trials and for the optimization of aNK cell-based therapies in patients with AML. The above data are taken from Boyiadzis M. et al., Cytotherapy, 2017; 19: 1225-1232.

### Example 2

This Example describes a representative method for treating patients with refractory or relapsed acute myeloid leukemia (AML) by administering NK-92 cells.

NK-92 cells are administered to patients with refractory or relapsed acute myeloid leukemia (AML) in an amount of about 1×10³ to about 1×10⁸ NK-92 cells per day for 21 days followed by seven days rest on a 28 day cycle. Patients diagnosed with AML are selected from those who are considered to be refractory to treatment after at least two cycles of treatment, or those who have relapsed after two cycles of treatment. The study is conducted in compliance with ESMO Clinical Practice Guidelines. Dosing occurs at approximately the same time each morning, where all doses are administered in the fasted state (no eating for at least two hours prior to dosing and two hours after dosing). Response is assessed at day 30 and monthly thereafter with serial peripheral blood counts and repeat bone marrow examinations. Patients are evaluated for blast clearance in the bone marrow to less than 5% of all nucleated cells, morphologically normal haematopoiesis and return of peripheral blood cell counts to normal levels. Patients with stable disease or better response are continued on therapy for a maximum of 12 months.

Blood sampling for analysis of pharmacokinetic parameters is performed on Days 1 and 28 according to the following sampling schedule: pre-dose, 0.25, 0.5, 0.75, 1, 1.5, 2, 2.5, 3, 4, 6, 8, 10, 12, 18, and 24 hours post-dose. Safety assessments are made by monitoring adverse events, vital signs, ECGs, clinical laboratory evaluations (blood chemistry, hematology, and lymphocyte phenotyping), and physical examination at specific times during the study. All patients are made available for toxicity testing. Patients who are available for response evaluation are evaluated. Patients on treatment are evaluated for response assessment where patients who achieve a complete response or a partial response are assessed. Patients who achieve stable disease (continued on treatment) are also assessed. An overall response rate in the evaluable patients is calculated, including the objective response rate defined as (complete response, partial response and stable disease).

The study results are expected to show that administering NK-92 cells is effective in treating refractory or relapsed leukemia.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### REFERENCES

[1] Childs R W, Carlsten M., Nat Rev Drug Discov 2015; 14(7):487-98

[2] Bjorkstrom N K et al., Nat Rev Immunol 16; 16(5):310-20.

[3] Ruggeri L et al., Science 2002; 295(5562):2097-100

[4] Ruggeri L et al., Blood 2007; 110(1):433-40

[5] Costello R T et al., Blood 2002; 99(10)3661-7

[6] Szczepanski M J et al., Cancer Immunol Immunother 2010; 59(1):73-9

[7] Tajima F, Kawatani T, Endo A, Kawasaki H., Leukemia 1996; 10(3):478-82

[8] Gong J H, Maki G, Klingemann H G et al., Leukemia 1994; 8(4):652-8

[9] Klingemann H G, Wong E, Maki G. et al., Biol Blood Marrow Transplant 1996; 2(2):68-75

[10] Yan Y et al., Clin Cancer Res 1998; 4(11): 2859-68

[11] Swerdlow S H, International Agency for Research on Cancer, World Health Organization. WHO classification of tumours of haematopoietic and lymphoid tissues. International Agency for Research on Cancer; 2008

[12] Arai S et al., Cytotherapy 2008; 10(6):625-32

[13] Tonn T et al., Cytotherapy 2013; 15(12):1563-70

[14] Cheson B D et al., J Clin Oncol 2003; 21(24):4642-9

[15] Knorr D A, Bachanova V, Verneris M R, Miller J S. et al., Semin Immunol 2014; 26(2):161-72

[16] Miller J S., Hematology 2013; 2013:247-53

[17] Klingemann H, Boissel L, Toneguzzo F., Front Immunol 2016; 7:91

[18] Suck G et al., Cancer Immunol Immunother 2016; 65(4):485-92

[19] Bachanova V et al., Blood 2014; 123(25):385563

[20] Curti A et al., Blood 2011; 118(12):3273-9

[21] Miller J S. et al., Blood 2005; 105(8):3051-7

[22] Rubnitz J E et al., J Clin Oncol 2010; 28(6):955-9

[23] Nannmark U et al., In Vivo 2000; 14(5):651-8

[24] Pegram H J et al., Cancer Immunol Immunother 2010; 59(8):1235-46

[25] Boyiadzis M et al., Blood 2016; 128:1609

[26] Boyiadzis M et al., Biol Blood Marrow Transplant 2008; 14(3):290-300

[27] Chik K W et al., J Pediatr Hematol Oncol 2003; 25(12):960-4

What is claimed is:

1. A method for treating remnant leukemia cells in a patient previously treated for leukemia, said method comprising administering to said patient NK-92 cells in an amount sufficient to kill a population of remnant leukemia cells remaining in the patient following conventional therapy for said leukemia.

2. The method of claim 1, wherein, prior to administering the NK-92 cells to the patient, said population of remnant leukemia cells are present in the patient at a level that is less than about 10% of the level of leukemia cells that was detected in the patient prior to the treatment for leukemia.

3. The method of claim 1, wherein said remnant leukemia cells comprise leukemic stem cells.

4. The method of claim 1, wherein said remnant leukemia cells comprise marrow cell precursors of lymphocytes, red blood cells, white blood cells or platelets.

5. The method of any one of claims 1-4, wherein said conventional therapy comprises one or more of chemotherapy, radiotherapy, hormone treatment or a bone marrow transplant.

6. The method of any one of claims 1-5, wherein said remnant leukemia cells are resistant to said conventional therapy.

7. A method for inhibiting a relapse of leukemia in a patient in recovery from leukemia, said method comprising administering to said patient one or more doses of NK-92 cells in an amount sufficient to inhibit relapse of leukemia in the patient.

8. The method of claim 7, wherein the relapse of leukemia in the patient is inhibited for at least about three months following the administration of the NK-92 cells.

9. The method of claim 7 or 8, wherein said conventional therapy comprises one or more of chemotherapy, radiotherapy, hormone treatment or a bone marrow transplant.

10. The method of any one of claims 1-9, wherein said leukemia is a lymphocytic leukemia or a myelogenous leukemia.

11. A pharmaceutical composition comprising a therapeutic dose of NK-92 cells for the treatment of leukemia.

12. A method for treating relapse of leukemia in a patient previously in recovery from leukemia, said method comprising administering to said patient one or more doses of NK-92 cells in an amount sufficient to treat said relapsed leukemia in the patient.

13. The method of claim 12, wherein said one or more doses of NK-92 cells is administered in combination with at least one anti-leukemic agent.

14. A method for treating leukemia in a patient who underwent conventional therapy for leukemia, said method comprising administering to said patient one or more doses of NK-92 cells in a therapeutic amount as an alternative to further conventional therapy.

15. The method of claim 14, wherein said administering to said patient of one or more doses of NK-92 cells serves as primary therapy after a relapse of leukemia in said patient.

16. The method of any one of the claims 1-15, wherein said NK-92 cells are unmodified NK-92 cells.

17. The method of any one of the claims 1-15, wherein said NK-92 cells are genetically modified NK-92 cells.

18. The method of any one of the claims 1-17, wherein said NK-92 cells are irradiated prior to being administered to the patient.

19. The method of any one of the claims 1-18, wherein said NK-92 cells secrete interleukin-2 (IL-2).

20. The pharmaceutical composition of claim 11 for use in the treatment of a leukemia or remnant leukemia.
